(19) United States
(12) Patent Application Publication
(10) Pub. No.: US 2024/0368561 A1
(43) Pub. Date: Nov. 7, 2024

(54) MEDIA COMPOSITION FOR INDUCED PLURIPOTENT STEM CELL DERIVED ENDOTHELIAL CELL GROWTH, MONOLAYER FORMATION, AND CELL-CELL CONTACT FORMATION

(71) Applicant: Lung Biotechnology PBC, Silver Spring, MD (US)

(72) Inventors: Barbara Nsiah, Manchester, NH (US); Jamie King, Bedford, NH (US); Sarah Gilpin, Silver Spring, MD (US)

(73) Assignee: Lung Biotechnology PBC, Silver Spring, MD (US)

(21) Appl. No.: 18/652,754

(22) Filed: May 1, 2024

Related U.S. Application Data
(60) Provisional application No. 63/463,722, filed on May 3, 2023.

(51) Int. Cl. C12N 5/074

(52) U.S. Cl. CPC C12N 5/0696; C12N 2501/105; C12N 2501/11; C12N 2501/115; C12N 2501/165; C12N 2501/30; C12N 2501/91; C12N 2501/999

(57) ABSTRACT

The present disclosure relates generally to, among other things, methods and compositions (e.g., culture media) for culturing iPS cells, such as iPS-endothelial cells (IPS-ECs). The present disclosure also provides iPS-EC cultures comprising iPS-ECs and a culture medium.

### CROSS REFERENCE TO RELATED APPLICATIONS

This application claims the benefit of and priority to U.S. Provisional Patent Application No. 63/463,722, filed on May 3, 2023, the contents of which are incorporated herein by reference in its entirety.

### FIELD

The present application relates generally to the field of culturing induced pluripotent stem (iPS) cell-derived cells, particularly to methods and compositions (e.g., culture media) for culturing iPS-derived cells, such as iPS-endothelial cells (iPS-ECs). The present disclosure also provides iPS-EC cultures comprising iPS-ECs and a culture medium.

### BACKGROUND

iPS-ECs can be used as research tools and also in tissue engineering or other clinical applications. For example, endothelial cells line the vessels of blood vessels, so iPS-ECs can be used in developed vascular grafts, such as 3-D printed vascular grafts. Often, however, in order for iPS-ECs to be most useful in research or clinical applications, the iPS-ECs must form a monolayer (e.g., comprising cell-cell contacts and/or junctions, e.g., adherens junctions, tight junctions). iPS-EC monolayers can serve to, for example, prevent blood clot formation during blood flow and selectively enable or prohibit certain fluids, molecules, and/or proteins (e.g., cytokines) from moving outside the vasculature lumen. Culturing iPS-EC monolayers (e.g., comprising cell-cell contacts and/or junctions) remains a challenge with current methods of culturing iPS-ECs.

To continue to advance technologies related to, for example, engineering vessels and/or organ grafts, it is important to be able to culture iPS-EC cells in monolayers (e.g., comprising cell-cell contacts and/or junctions). Accordingly, there remains a need for improved methods and compositions for culturing iPS-ECs in a monolayer (e.g., comprising cell-cell contacts and junctions).

### SUMMARY OF THE INVENTION

The present disclosure provides, among other things, improved methods of culturing induced pluripotent derived endothelial cells (iPS-ECs) in vitro, compositions for same (e.g., culture medium), and iPS-EC cultures comprising iPS-ECs and a culture medium.

In one aspect, the present disclosure provides a method of culturing induced pluripotent stem cell derived endothelial cells (iPS-ECs) in vitro comprising culturing the iPS-ECs with a culture medium comprising one or more growth factors selected from Vascular Endothelial Growth Factor (VEGF) and basic Fibroblast Growth Factor (bFGF).

In some embodiments, the culture medium comprises about 30 to about 40 ng/ml VEGF.

In some embodiments, the culture medium comprises about 20 to about 40 ng/ml bFGF.

In some embodiments, the culture medium comprises one or more additional reagents selected from the group consisting of: serum, hydrocortisone, Epidermal Growth Factor (EGF), Insulin-like Growth Factor (IGF), an anticoagulant, and an antibiotic.

In some embodiments, the culture medium comprises serum at a concentration of about 2% by volume. In some embodiments, the serum comprises fetal bovine serum.

In some embodiments, the culture medium comprises hydrocortisone at a concentration of about 0.2 μg/mL.

In some embodiments, the culture medium comprises EGF at a concentration of about 5 ng/mL.

In some embodiments, the culture medium comprises IGF at a concentration of about 20 ng/mL.

In some embodiments, the anticoagulant comprises heparin or ascorbic acid. In some embodiments, the culture medium comprises ascorbic acid at a concentration of about 1 μg/mL. In some embodiments, the culture medium comprises heparin at a concentration of about 22.5 μg/mL.

In some embodiments, the antibiotic comprises one or more of penicillin, streptomycin, gentamicin, amphotericin, or any combination thereof. In some embodiments, the culture medium comprises penicillin and streptomycin at an amount of about 1% by volume. In some embodiments, the culture medium comprises gentamicin and amphotericin at a concentration of about 30 μg/ml gentamicin and about 15 ng/ml amphotericin.

In some embodiments, the culture medium comprises a base media for growing endothelial cells.

In some embodiments, the culture medium is exchanged with a frequency of about every day, every other day, every three days, every five days, or every week.

In some embodiments, the iPS-ECs are plated for culturing at a density of about 50,000 cells/cm², about 25,000 cells/cm², or about 12,500 cells/cm².

In some embodiments, the iPS-ECs form one or more of a monolayer, cell-cell contacts, and tight junctions.

In one aspect, the present disclosure provides, an iPS-EC culture comprising iPS-ECs and a culture medium comprising VEGF and bFGF.

In some embodiments, the culture medium comprises about 30 to about 40 ng/ml VEGF.

In some embodiments, the culture medium comprises about 20 to about 40 ng/ml bFGF.

### BRIEF DESCRIPTION OF THE DRAWINGS

FIG. 1A-1C shows growth of iPS-EC cells on plastic in Media A, Media B, and Media C after 1 day in culture (FIG. 1A), 4 days in culture (FIG. 1B), and 6 days in culture (FIG. 1C).

FIG. 2A-2B shows glucose consumption of iPS-ECs plated on either plastic and grown in either Media A, B, or C after 1, 2, 4, or 7 days in culture (FIG. 2A) or hydrogel discs and grown in either of Media A, B, or C after 1, 4, or 7 days in culture (FIG. 2B)

FIG. 3A-3B shows lactate production of iPS-ECs plated on either plastic and grown in either Media A, B, or C after 1, 2, 4, or 7 days in culture (FIG. 3A) or hydrogel discs and grown in either of Media A, B, or C after 1, 4, or 7 days in culture (FIG. 3B).

FIG. 4A-4C shows fluorescent microscope images of iPS-ECs grown on plastic in either of Media B (FIG. 4A), Media A (FIG. 4B), or Media C (FIG. 4C) after 2 days in culture. DAPI (blue), Phalloidin (Green), Ve-Cadherin (Red), and Ki-67 (yellow).

FIG. 5 shows fluorescent microscopy images of iPS-ECs grown in either of Media C, Media D, or a commercially available culture media, Media E, after 4 days in culture. DAPI (blue), Phalloidin (green), Ve-Cadherin (red), and Ki-67 (yellow).

FIG. 6 shows fluorescent microscopy images of iPS-ECs grown in either of Media C, Media D, or a commercially available culture media, Media E, after 7 days in culture. DAPI (blue), Phalloidin (green), Ve-Cadherin (red), and Ki-67 (yellow).

FIG. 7 shows fluorescent microscopy images of iPS-EC cellularized rabbit lobes. DAPI (blue), Phalloidin (green).

### DETAILED DESCRIPTION

It is to be appreciated that certain aspects, modes, embodiments, variations and features of the present methods are described below in various levels of detail in order to provide a substantial understanding of the present technology.

The present disclosure is not to be limited in terms of the particular embodiments described in this application, which are intended as single illustrations of individual aspects of the disclosure. All the various embodiments of the present disclosure will not be described herein. Many modifications and variations of the disclosure can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled.

It is to be understood that the present disclosure is not limited to particular uses, methods, reagents, compounds, compositions or biological systems, which can, of course, vary. It is also to be understood that the terminology used herein is for the purpose of describing particular embodiments only, and is not intended to be limiting.

In practicing the present methods, many conventional techniques in molecular biology, protein biochemistry, cell biology, microbiology and recombinant DNA are used. Sec, e.g., Sambrook and Russell eds. (2001) Molecular Cloning: A Laboratory Manual, 3rd edition; the series Ausubel et al. eds. (2007) Current Protocols in Molecular Biology, the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach, Harlow and Lane eds. (1999) Antibodies, A Laboratory Manual, Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization, Anderson (1999) Nucleic Acid Hybridization, Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology.

iPS-ECs are important tools, not only for biomedical research, but also for use in engineering vessels and/or grafts (e.g., organ grafts), including, for example, 3-dimensionally (3D) printed grafts. iPS-ECs can be used to generate cellularized vasculature of grafts (e.g., 3D printed grafts) for both autologous and allogeneic applications. iPS-ECs can also function as a non-thromogenic barrier within, for example, arterial, venous, and capillaries of vasculature networks. However, in order for iPS-ECs to contribute to the vascularization of engineered vessels and/or grafts (e.g., 3D printed grafts), the iPS-ECs must form a continuous monolayer (e.g., comprising cell-cell contacts and/or junctions (e.g., adherens junctions, tight junctions)). iPS-ECs monolayers can also serve to, for example, prevent blood clot formation during blood flow and selectively enable or prohibit certain fluids, molecules, and/or proteins (e.g., cytokines) from moving outside the vasculature lumen. Culturing iPS-EC monolayers (e.g., comprising cell-cell contacts and/or junctions) remains a challenge with current methods of culturing iPS-ECs.

The present disclosure relates generally to, among other things, methods and compositions (e.g., culture media) for culturing iPS cells, such as iPS-endothelial cells (iPS-ECs). The present disclosure also provides iPS-EC cultures comprising iPS-ECs and a culture medium.

### Definitions

Unless defined otherwise, all technical and scientific terms used herein have the meaning commonly understood by a person skilled in the art to which this disclosure belongs. As used herein, the following terms have the meanings ascribed to them below, unless specified otherwise. The terminology used herein is for the purpose of describing particular embodiments only and is not intended to be limiting of the disclosure.

As used herein, the single forms “a”, “an”, and “the” are intended to include the plural forms as well, unless the context clearly indicates otherwise.

As used here, the term “about,” when used to modify a numerical value, indicates that deviations of up to 10% above and below the numerical value, including the numerical value, remain within the intended meaning of the recited value. For example, “about 10” should be understood as both “10” and “9-11”.

As used herein, the term “allogeneic” refers to a cell or tissue which is derived from one subject (e.g., a donor subject) of the same species as the subject in need thereof (e.g., in need of a graft) who is not genetically identical. This is distinct from “autologous,” where the subject (e.g., donor subject) and the subject in need thereof (e.g., in need of a graft) are genetically identical (e.g., are the same subject).

As used herein, the term “base medium” or “base media” refers to liquid medium for cell culture comprising a suitable carbon source such as a sugar, lipids, vitamins, and amino acids and a buffering system. The medium contains levels and/or ratios of salts and/or nutrients needed to support cell growth. The medium may be in dry or liquid form and may be prepared from a plurality of separate stock compositions (e.g., each independently existing in dry or solution form) that can be combined prior to use. For example, the medium may be prepared from two, three, four, or more stock compositions (each independently in dry or solution form), and where necessary mixed with aqueous diluent prior to use to give a 1× medium formulation.

As used herein, the term “cell population” or “population of cells” refers to a group of at least two cells expressing similar or different phenotypes (e.g., iPS-ECs). In non-limiting examples, a cell population can include at least about 10, at least about 100, at least about 200, at least about 300, at least about 400, at least about 500, at least about 600, at least about 700, at least about 800, at least about 900, at least about 1000 cells, at least about 10,000 cells, at least about 100,000 cells, at least about 1×10⁶ cells, at least about 1×10⁷ cells, at least about 1×10⁸ cells, at least about 1×10⁹ cells, at least about 1×10¹⁰ cells, at least about 1×10¹¹ cells, at least about 1×10¹² cells, or more cells expressing similar or different phenotypes.

As used herein, the term “comprising” is intended to mean that the compositions and methods include the recited elements, but not excluding others. “Consisting essentially of” when used to define compositions and methods, shall mean excluding other elements of any essential significance to the composition or method. “Consisting of” shall mean excluding more than trace elements of other ingredients for claimed compositions and substantial method steps. Embodiments defined by each of these transition terms are within the scope of this disclosure. Accordingly, it is intended that the methods and compositions can include additional steps and components (comprising) or alternatively including steps and compositions of no significance (consisting essentially of) or alternatively, intending only the stated method steps or compositions (consisting of).

As used herein, a “control” is an alternative sample used in an experiment for comparison purpose. A control can be “positive” or “negative.” For example, where the purpose of the experiment is to determine a correlation of the efficacy of a therapeutic agent for the treatment for a particular type of disease, a positive control (a composition known to exhibit the desired therapeutic effect) and a negative control (a subject or a sample that does not receive the therapy or receives a placebo) are typically employed.

As used herein, the term “donor subject” refers to the source of a cell or tissue (e.g., pluripotent stem cells that can be differentiated into iPS-ECs). A donor subject can be any suitable animal, including, for example, humans, cows, sheep, mouse, rat, non-human primates, horse, dog, cat, etc.

As used herein, the term “edited iPS-EC” refers to an iPS-EC, which has been modified to change at least one expression product of at least one gene at some point in the development and/or culturing of the cell. A modification can be introduced using any suitable technique including, but not limited to, CRISPR-Cas and dominant-negative constructs. An iPS-EC can be edited at a time point before it has differentiated into an iPS-EC. e.g., at a precursor stage, at a stem cell stage, or after differentiation. An edited iPS-EC can be compared to a non-edited iPS-EC (an iPS-EC produced by differentiating a pluripotent stem cell, in which the pluripotent stem cell and/or iPS-EC cell do not have modifications, e.g., genetic modifications).

As used herein, the term “graft” refers to a portion of tissue or a collection of cells that is suitable for implantation or transplantation. It is intended that the term encompass any graft material and types, including but not limited to autologous, allogeneic, avascular, accordion, autodermic, autoepidermic, bone, fascicular, full-thickness, heterologous, heteroplastic, xenografts, nerve, muscle, tendon, ligament, synthetic (e.g., artificial), and other suitable grafts, including grafts obtained from biological material grown in vitro. Artificial grafts may be produced, for example, by 3D printing. A graft can be a portion of an organ, or whole organ, or an approximation of an organ or portion thereof. Grafts include vascular grafts. The term “grafting” refers to the process of implanting or transplanting a graft.

As used herein, the term “pharmaceutically-acceptable carrier” is intended to include any and all solvents, dispersion media, coatings, antibacterial and antifungal compounds, isotonic and absorption delaying compounds, and the like, compatible with pharmaceutical administration. Pharmaceutically-acceptable carriers and their formulations are known to one skilled in the art and are described, for example, in Remington's Pharmaceutical Sciences (20th edition, ed. A. Gennaro, 2000, Lippincott, Williams & Wilkins, Philadelphia, PA.).

As used herein, the terms “subject,” “individual,” or “patient” are used interchangeably and refer to an individual organism, a vertebrate, or a mammal and may include humans, non-human primates, rodents, and the like (e.g., which is to be the recipient of a particular treatment, or from whom cells are harvested). In certain embodiments, the individual, patient or subject is a human.

Methods of Culturing iPS-ECs

Methods of culturing iPS-ECs described herein utilize conditions that can result in the formation of iPS-EC monolayers, e.g., comprising cell-cell contacts and/or junctions (e.g., adherens junctions, tight junctions). Methods of culturing iPS-ECs of the present disclosure can be conducted in vitro and comprise culturing the iPS-ECs with a culture medium comprising one or more growth factors selected from Vascular Endothelial Growth Factor (VEGF) and basic Fibroblast Growth Factor (bFGF).

In some embodiments, the culture medium comprises about 1 to about 100 ng/ml VEGF, about 1 to about 90 ng/mL VEGF, about 1 to about 80 ng/mL VEGF, about 1 to about 70 ng/mL VEGF, about 1 to about 60 ng/mL VEGF, about 1 to about 50 ng/mL VEGF, about 1 to about 40 ng/mL VEGF, about 5 to about 100 ng/mL VEGF, about 5 to about 90 ng/mL VEGF, about 5 to about 80 ng/mL VEGF, about 5 to about 70 ng/mL VEGF, about 5 to about 60 ng/mL VEGF, about 5 to about 50 ng/mL VEGF, about 5 to about 40 ng/mL VEGF, about 10 to about 100 ng/mL VEGF, about 10 to about 90 ng/mL VEGF, about 10 to about 80 ng/mL VEGF, about 10 to about 70 ng/mL VEGF, about 10 to about 60 ng/ml VEGF, about 10 to about 50 ng/mL VEGF, or about 10 to about 40 ng/mL VEGF.

In some embodiments, the culture medium comprises about 1 to about 50 ng/ml VEGF. In some embodiments, the culture medium comprises about 10 to about 50 ng/ml VEGF. In some embodiments, the culture medium comprises about 10 to about 40 ng/ml VEGF. In some embodiments, the culture medium comprises about 20 to about 40 ng/ml VEGF. In some embodiments, the culture medium comprises about 30 to about 40 ng/ml VEGF.

In some embodiments, the culture medium comprises about 1 ng/mL VEGF. In some embodiments, the culture medium comprises about 5 ng/mL VEGF. In some embodiments, the culture medium comprises about 10 ng/mL VEGF. In some embodiments, the culture medium comprises about 20 ng/mL VEGF. In some embodiments, the culture medium comprises about 30 ng/mL VEGF. In some embodiments, the culture medium comprises about 40 ng/mL VEGF. In some embodiments, the culture medium comprises about 50 ng/mL VEGF. In some embodiments, the culture medium comprises about 60 ng/mL VEGF.

In some embodiments, the culture medium comprises about 10 to about 100 ng/ml bFGF, about 10 to about 90 ng/mL bFGF, about 10 to about 80 ng/ml bFGF, about 10 to about 70 ng/mL bFGF, about 10 to about 60 ng/mL bFGF, about 10 to about 50 ng/mL bFGF, about 10 to about 40 ng/mL bFGF, about 20 to about 100 ng/mL bFGF, about 20 to about 90 ng/mL bFGF, about 20 to about 80 ng/mL bFGF, about 20 to about 70 ng/mL bFGF, about 20 to about 60 ng/mL bFGF, about 20 to about 50 ng/mL bFGF, or about 20 to about 40 ng/ml bFGF.

In some embodiments, the culture medium comprises about 10 to about 50 ng/ml bFGF. In some embodiments, the culture medium comprises about 20 to about 50 ng/ml bFGF. In some embodiments, the culture medium comprises about 10 to about 40 ng/ml bFGF. In some embodiments, the culture medium comprises about 20 to about 40 ng/ml bFGF.

In some embodiments, the culture medium comprises about 10 ng/mL bFGF. In some embodiments, the culture medium comprises about 20 ng/mL bFGF. In some embodiments, the culture medium comprises about 30 ng/mL bFGF. In some embodiments, the culture medium comprises about 40 ng/mL bFGF. In some embodiments, the culture medium comprises about 50 ng/mL bFGF. In some embodiments, the culture medium comprises about 60 ng/ml bFGF.

Culture medium for use in accordance with methods described herein can comprise a base media (e.g., a base media for growing endothelial cells). Base media can include, for example and without limitation, DMEM, alphaMEM, EGM™-2 Endothelial Cell Growth Medium-2, EGM™-2 MV Microvascular Endothelial Cell Growth Medium-2, EBM™-2 Endothelial Cell Growth Basal Medium-2, EGM™ Endothelial Cell Growth Medium, and Human Endothelial-Serum Free media (HE-SFM).

### General Culturing Methods

Methods of culturing iPS-ECs of the present disclosure can comprise plating (also referred to herein as “seeding”) the iPS-ECs for culturing at a certain cell number (e.g., total number of cells in the culture at plating) and/or density (e.g., number of cells in the culture at plating per surface area of the culture vessel).

In some embodiments, iPS-ECs are plated for culturing at a cell number of about 1,000 to about 10,000 cells, about 2,500 to about 10,000 cells, about 5,000 to about 10,000 cells, about 1,000 to about 100,000 cells, about 5,000 to about 100,000 cells, about 10,000 to about 100,000 cells, about 20,000 to about 100,000 cells, about 50,000 to about 100,000 cells, about 100,000 to about 5 million cells, about 100,000 to about 4.5 million cells, about 100,000 to about 4 million cells, about 100,000 to about 3.5 million cells, about 100,000 to about 3 million cells, about 100,000 to about 2.5 million cells, about 100,000 to about 2 million cells, about 100,000 to about 1.5 million cells, about 100,000 to about 1 million cells, about 100,000 to about 500,000 cells, or about 100,000 to about 250,000 cells.

In some embodiments, iPS-ECs are plated for culturing at a cell number of about 1,000 to about 10,000 cells. In some embodiments, iPS-ECs are plated for culturing at a cell number of about 2,500 to about 10,000 cells. In some embodiments, iPS-ECs are plated for culturing at a cell number of about 5,000 to about 10,000 cells. In some embodiments, iPS-ECs are plated for culturing at a cell number of about 10,000 to about 100,000 cells.

In some embodiments, iPS-ECs are plated for culturing at a cell number of about 1,000 cells. In some embodiments, iPS-ECs are plated for culturing at a cell number of about 5,000 cells. In some embodiments, iPS-ECs are plated for culturing at a cell number of about 10,000 cells. In some embodiments, iPS-ECs are plated for culturing at a cell number of about 20,000 cells. In some embodiments, iPS-ECs are plated for culturing at a cell number of about 50,000 cells. In some embodiments, iPS-ECs are plated for culturing at a cell number of about 100,000 cells. In some embodiments, iPS-ECs are plated for culturing at a cell number of about 250,000 cells. In some embodiments, iPS-ECs are plated for culturing at a cell number of about 500,000 cells. In some embodiments, iPS-ECs are plated for culturing at a cell number of about 1 million cells. In some embodiments, iPS-ECs are plated for culturing at a cell number of about 2 million cells. In some embodiments, iPS-ECs are plated for culturing at a cell number of about 5 million cells.

In some embodiments, iPS-ECs are plated for culturing at a density of about 1,000 to about 10,000 cells/cm², about 2,500 to about 10,000 cells/cm², about 5,000 to about 10,000 cells/cm², about 10,000 to about 100,000 cells/cm², about 20,000 to about 100,000 cells/cm², about 50,000 to about 100,000 cells/cm², about 10,000 to about 150,000 cells/cm², about 12,500 to about 150,000 cells/cm², about 25,000 to about 150,000 cells/cm², about 50,000 to about 150,000 cells/cm², about 12,500 to about 100,000 cells/cm², about 12,500 to about 75,000 cells/cm², about 12,500 to about 50,000 cells/cm², about 25,000 to about 100,000 cells/cm², about 25,000 to about 75,000 cells/cm², about 25,000 to about 50,000 cells/cm², about 50,000 to about 100,000 cells/cm², or about 50,000 to about 75,000 cells/cm².

In some embodiments, iPS-ECs are plated for culturing at a density of about 1,000 to about 10,000 cells/cm². In some embodiments, iPS-ECs are plated for culturing at a density of about 2,500 to about 10,000 cells/cm². In some embodiments, iPS-ECs are plated for culturing at a density of about 5,000 to about 10,000 cells/cm². In some embodiments, iPS-ECs are plated for culturing at a density of about 10,000 to about 100,000 cells/cm².

In some embodiments, iPS-ECs are plate for culturing at a density of about 1,000 cells/cm². In some embodiments, iPS-ECs are plate for culturing at a density of about 2,500 cells/cm². In some embodiments, iPS-ECs are plate for culturing at a density of about 5,000 cells/cm². In some embodiments, iPS-ECs are plate for culturing at a density of about 10,000 cells/cm². In some embodiments, iPS-ECs are plate for culturing at a density of about 12,500 cells/cm². In some embodiments, iPS-ECs are plate for culturing at a density of about 25,000 cells/cm². In some embodiments, iPS-ECs are plate for culturing at a density of about 50,000 cells/cm². In some embodiments, iPS-ECs are plate for culturing at a density of about 75,000 cells/cm². In some embodiments, iPS-ECs are plate for culturing at a density of about 100,000 cells/cm².

Methods of culturing iPS-ECs of the present disclosure can comprise adding additional culture medium to the iPS-ECs and/or exchanging the culture medium.

Methods of culturing iPS-ECs of the present disclosure can comprise adding additional culture medium to the iPS-ECs. In some embodiments, the additional culture medium is added to the iPS-ECs with a frequency of about every day, every other day, every three days, every five days, or every week. In some embodiments, the frequency is about every day. In some embodiments, the frequency is about every other day. In some embodiments, the frequency is about every three days. In some embodiments, the frequency is about every five days. In some embodiments, the frequency is about every week.

In some embodiments, the additional culture medium is added to the iPS-ECs at a volume to maintain a consistent volume of culture medium while culturing (e.g., to off-set evaporation of the initial culture media added to the iPS-ECs over the course of culturing). In some embodiments, the additional culture medium is added to the iPS-ECs at a volume of about 10%, about 20%, about 30%, about 40%, about 50%, about 60%, about 70%, about 80%, about 90%, or about 100% of the initial volume of culture medium added to the iPS-ECs.

Methods of culturing iPS-ECs of the present disclosure can comprise exchanging the culture medium (e.g., removing the total volume of culture medium in the culture and replacing it with fresh culture medium at a certain volume). In some embodiments, the culture medium is exchanged with a frequency of about every day, every other day, every three days, every five days, or every week. In some embodiments, the frequency is about every day. In some embodiments, the frequency is about every other day. In some embodiments, the frequency is about every three days. In some embodiments, the frequency is about every five days. In some embodiments, the frequency is about every week.

In some embodiments, the culture medium is exchanged with at a volume that is about equivalent to the volume that was removed and/or the initial volume of culture medium added to the iPS-ECs. In some embodiments, the culture medium is exchanged at a volume of about 10%, about 20%, about 30%, about 40%, about 50%, about 60%, about 70%, about 80%, about 90%, about 100%, about 110%, about 125%, about 150%, or about 200% of the initial volume of culture medium added to the iPS-ECs.

Methods of culturing iPS-ECs of the present disclosure can comprise culturing the iPS-ECs at a certain temperature and/or humidity. It will be readily apparent to one or ordinary skill in the art reading the present disclosure how to select an appropriate temperature and/or humidity for use with methods of culturing described herein. For example, iPS-ECs can be cultured in accordance with technologies described herein at about 37° C., a humidity of about 85-95%, and 5% CO₂ and 20% O₂.

Methods of the present disclosure may be carried out in any suitable cell culture vessel and/or cell culture container. The surface of a cell culture vessel and/or container may be coated with cell surface coatings (e.g., adhesion-promoting matrices and/or substrates). Non-limiting examples of adhesion-promoting matrices and/or substrates include, poly-D-Lysine, Poly-L-Lysine, Poly-Ornithine, gelatin, collagens, fibronectins, laminins and laminin derived peptides (e.g., AG73), vitronectin, osteopontin, polypeptide comprising the amino acid sequence PHSRNKRGDS, BM Binder, mixtures of naturally occurring cell line-produced matrices such as Matrigel™, RGD-containing polypeptides, and the like.

### Additional Reagents

Culture medium (e.g., for use in accordance with methods described herein) can also comprise additional reagents. Additional reagents can include, for example and without limitation, serum, hormones, growth factors, anticoagulants, antibiotics, or any combination thereof

In some embodiments, culture medium comprises serum. Serum can be an important source of, for example, growth and adhesion factors, hormones, lipids, and/or minerals for the culture of cells (e.g., methods of culturing iPS-ECs as described herein). Serum can also serve as a carrier for lipids, enzymes, micronutrients, and/or trace elements into cells (e.g., iPS-ECs). Sera may include, for example and without limitation, bovine serum (e.g., fetal bovine serum), rabbit serum, sheep serum, goat serum, procine serum, chicken serum, horse serum, and serum replacements, such as human platelet lysate. In some embodiments, the culture medium comprises fetal bovine serum.

In some embodiments, the culture medium comprises serum at a concentration of about 1%, about 2%, about 3%, about 4%, about 5%, about 6%, about 7%, about 8%, about 9%, or about 10%. In some embodiments, the culture medium comprises serum at a concentration of about 1%. In some embodiments, the culture medium comprises serum at a concentration of about 2%. In some embodiments, the culture medium comprises serum at a concentration of about 5%.

In some embodiments, culture medium comprises hormones (including, e.g., hormone-like substances). Hormones are a class of signaling molecules used by multicellular organisms to organize, coordinate, and/or control the functions of their cells and tissues. Hormones can further support and/or stimulate cellular proliferation of cells in culture. Hormones can include, for example, hydrocortisone, prostaglandins, insulin, glucagon, corticosteroids, vasopressin, thyroid hormones, parathyroid hormone, growth hormone, and glandotropic hormones. In some embodiments, the culture medium comprises one or more of hydrocortisone, prostaglandins, insulin, glucagon, corticosteroids, vasopressin, thyroid hormones, parathyroid hormone, growth hormone, glandotropic hormones, or any combination thereof.

Hydrocortisone (also referred to as cortisol) is an endogenous glucocorticoid, which can be secreted by the adrenal gland. Hydrocortisone can interact with glucocorticoid receptors and regulate various signaling pathways and has been associated with, for example, the induction of energy metabolism and local glucose consumption. In some embodiments, the culture medium comprises hydrocortisone.

In some embodiments, the culture medium comprises hydrocortisone at a concentration of about 0.1 μg/mL, about 0.2 μg/mL, about 0.3 μg/mL, about 0.5 μg/mL, or about 1.0 μg/mL. In some embodiments, the culture medium comprises hydrocortisone at a concentration of about 0.1 μg/mL. In some embodiments, the culture medium comprises hydrocortisone at a concentration of about 0.2 μg/mL. In some embodiments, the culture medium comprises hydrocortisone at a concentration of about 0.5 μg/mL. In some embodiments, the culture medium comprises hydrocortisone at a concentration of about 1.0 μg/mL.

Prostaglandins are hormone-like substances that play important roles in a number of biological functions, such as inflammation, pain, and uterine contractions. In some embodiments, the culture medium comprises one or more prostaglandins.

In some embodiments, the culture medium comprises one or more prostaglandins at a concentration of about 1 ng/mL, about 1.5 ng/ml, about 2 ng/ml, about 3 ng/ml, or about 5 ng/mL. In some embodiments, the culture medium comprises one or more prostaglandins at a concentration of about 1 ng/mL. In some embodiments, the culture medium comprises one or more prostaglandins at a concentration of about 1.5 ng/mL. In some embodiments, the culture medium comprises one or more prostaglandins at a concentration of about 2 ng/ml. In some embodiments, the culture medium comprises one or more prostaglandins at a concentration of about 3 ng/mL. In some embodiments, the culture medium comprises one or more prostaglandins at a concentration of about 5 ng/mL.

Insulin is a peptide hormone produced by B cells of pancreatic islets and maintains normal blood glucose levels by facilitating cellular glucose uptake, carbohydrate, lipid and protein metabolism and promoting cell division and growth through its mitogenic effects. In some embodiments, the culture medium comprises insulin.

In some embodiments, the culture medium comprises insulin at a concentration of about 1 μg/mL, about 2 μg/mL, about 3 μg/mL, about 4 μg/mL, about 5 μg/mL, about 6 μg/mL, about 8 μg/mL, or about 10 μg/mL. In some embodiments, the culture medium comprises insulin at a concentration of about 1 μg/mL. In some embodiments, the culture medium comprises insulin at a concentration of about 2 μg/mL. In some embodiments, the culture medium comprises insulin at a concentration of about 5 g/mL. In some embodiments, the culture medium comprises insulin at a concentration of about 10 μg/mL.

Glucagon is a peptide hormone secreted by a cells in the pancreas. Its release is controlled by hypothalamus when glucose levels in the blood decreases. In some embodiments, the culture medium comprises glucagon.

In some embodiments, the culture medium comprises glucagon at a concentration of about 0.3 μg/mL, about 0.5 μg/mL, about 1 μg/mL, about 2 μg/mL, about 5 μg/mL, or about 10 μg/mL. In some embodiments, the culture medium comprises glucagon at a concentration of about 0.3 μg/mL. In some embodiments, the culture medium comprises glucagon at a concentration of about 0.5 μg/mL. In some embodiments, the culture medium comprises glucagon at a concentration of about 1 μg/mL. In some embodiments, the culture medium comprises glucagon at a concentration of about 2 μg/mL. In some embodiments, the culture medium comprises glucagon at a concentration of about 5 μg/mL. In some embodiments, the culture medium comprises glucagon at a concentration of about 10 μg/mL.

Corticosteroids are a class of steroid hormones released by the adrenal cortex, which includes, for example, glucocorticoids and mineralocorticoids. In some embodiments, the culture medium comprises one or more corticosteroids. One of ordinary skill in the art would readily understand that the concentration of corticosteroid to be used in the cell culture medium would be dependent on the particular corticosteroid(s) to be utilized (e.g., a glucocorticoid, a mineralocorticoid). Determination of an appropriate concentration of a particular corticosteroid(s) to be added to and/or included in the culture medium is well within the level of one of ordinary skill.

Vasopressin or antidiuretic hormone (ADH) or arginine vasopressin (AVP) is a nonapeptide synthesized in the hypothalamus, known to play important roles in the control of the body's osmotic balance, blood pressure regulation, sodium homeostasis, and kidney functioning. In some embodiments, the culture medium comprises vasopressin.

In some embodiments, the culture medium comprises vasopressin at a concentration of about 1 pg/mL, about 2 pg/mL, about 5 pg/mL, about 10 pg/mL, about 15 pg/mL, or about 20 pg/mL. In some embodiments, the culture medium comprises vasopressin at a concentration of about 1 pg/mL. In some embodiments, the culture medium comprises vasopressin at a concentration of about 2 pg/mL. In some embodiments, the culture medium comprises vasopressin at a concentration of about 5 pg/mL. In some embodiments, the culture medium comprises vasopressin at a concentration of about 10 pg/mL. In some embodiments, the culture medium comprises vasopressin at a concentration of about 15 pg/mL. In some embodiments, the culture medium comprises vasopressin at a concentration of about 20 pg/mL.

Thyroid hormones (e.g., T3, T4) are released by the thyroid gland and play important roles in regulation of weight, energy levels, internal temperature, metabolism, growth of skin, hair, and nails, and the endocrine system. In some embodiments, the culture medium comprises one or more thyroid hormones. One of ordinary skill in the art would readily understand that the concentration of thyroid hormone to be used in the cell culture medium would be dependent on the particular thyroid hormone(s) to be utilized (e.g., T3, T4). Determination of an appropriate concentration of a particular thyroid hormone(s) to be added to and/or included in the culture medium is well within the level of one of ordinary skill.

Parathyroid hormone is also produced by the thyroid gland and helps to maintain the balance of calcium in the blood stream and tissues that depend on calcium for proper function (e.g., nerves, muscles). In some embodiments, the culture medium comprises parathyroid hormone.

In some embodiments, the culture medium comprises parathyroid hormone at a concentration of about 10 pg/mL, about 15 pg/mL, about 20 pg/mL, about 25 pg/mL, about 30 pg/mL, about 40 pg/mL or about 50 pg/mL. In some embodiments, the culture medium comprises parathyroid hormone at a concentration of about 10 pg/mL. In some embodiments, the culture medium comprises parathyroid hormone at a concentration of about 15 pg/mL. In some embodiments, the culture medium comprises parathyroid hormone at a concentration of about 20 pg/mL. In some embodiments, the culture medium comprises parathyroid hormone at a concentration of about 25 pg/mL. In some embodiments, the culture medium comprises parathyroid hormone at a concentration of about 30 pg/mL. In some embodiments, the culture medium comprises parathyroid hormone at a concentration of about 40 pg/mL. In some embodiments, the culture medium comprises parathyroid hormone at a concentration of about 50 pg/mL.

Growth hormone, such as human growth hormone (HGH), is a single-chain polypeptide produced by somatotropic cells within the anterior pituitary gland and is important in growth regulation during childhood and certain basal metabolic functions. In some embodiments, the culture medium comprises growth hormone.

In some embodiments, the culture medium comprises growth hormone at a concentration of about 0.01 ng/mL, about 0.02 ng/mL, about 0.05 ng/ml or about 1 ng/mL. In some embodiments, the culture medium comprises growth hormone at a concentration of about 0.01 ng/mL. In some embodiments, the culture medium comprises growth hormone at a concentration of about 0.02 ng/mL. In some embodiments, the culture medium comprises growth hormone at a concentration of about 0.05 ng/mL. In some embodiments, the culture medium comprises growth hormone at a concentration of about 1 ng/mL.

Glandotropic hormones, such as thyroid-stimulating hormone (TSH), adrenocorticotropic hormone (ACTH), follicle-stimulating hormone (FSH), luteinizing hormone (LH), are primarily produced and secreted by the anterior pituitary and affect the secretion of other endocrine glands. In some embodiments, the culture medium comprises one or more glandotropic hormones. One of ordinary skill in the art would readily understand that the concentration of glandotropic hormone to be used in the cell culture medium would be dependent on the particular glandotropic hormone(s) to be utilized (e.g., TSH, LH). Determination of an appropriate concentration of a particular glandotropic hormone(s) to be added to and/or included in the culture medium is well within the level of one of ordinary skill.

In some embodiments, the culture medium comprises one or more growth factors. Growth factors are molecules capable of stimulating a variety of cellular processes, including, for example, cell proliferation and cell migration. Examples of growth factors include, without limitation, Bone Morphogenetic Proteins (BMPs), Epidermal Growth Factor (EGF), Endothelial Cell Growth Factors (ECGFs), Insulin-like growth factors (IGF), and cytokines.

The culture medium can comprise one or more growth factors (e.g., for use in accordance with methods described herein) at a concentration of about 1 to about 50 ng/ml, about 2 to about 50 ng/ml, about 5 to about 50 ng/mL, about 10 to about 50 ng/ml, about 20 to about 50 ng/ml, about 1 to about 40 ng/ml, about 2 to about 40 ng/ml, about 5 to about 40 ng/mL, about 10 to about 40 ng/ml, about 20 to about 40 ng/ml, about 1 to about 20 ng/mL, about 2 to about 20 ng/mL, or about 5 to about 20 ng/mL. In some embodiments, the culture medium comprises one or more growth factors at a concentration of about 1 ng/mL. In some embodiments, the culture medium comprises one or more growth factors at a concentration of about 5 ng/mL. In some embodiments, the culture medium comprises one or more growth factors at a concentration of about 20 ng/mL.

In some embodiments, the culture medium comprises EGF. In some such embodiments, the culture medium comprises EGF at a concentration of about 1 to about 10 ng/mL. In some such embodiments, the culture medium comprises EGF at a concentration of about 1 ng/mL. In some such embodiments, the culture medium comprises EGF at a concentration of about 2 ng/mL. In some such embodiments, the culture medium comprises EGF at a concentration of about 5 ng/mL. In some such embodiments, the culture medium comprises EGF at a concentration of about 10 ng/mL.

In some embodiments, the culture medium comprises IGF. In some such embodiments, the culture medium comprises IGF at a concentration of about 15 to about 25 ng/mL. In some such embodiments, the culture medium comprises IGF at a concentration of about 15 ng/mL. In some such embodiments, the culture medium comprises IGF at a concentration of about 20 ng/mL. In some such embodiments, the culture medium comprises IGF at a concentration of about 25 ng/mL.

In some embodiments, the culture medium comprises one or more anticoagulants. Anticoagulants can include, for example, and without limitation, heparin or ascorbic acid.

In some embodiments, the culture medium comprises ascorbic acid. Ascorbic acid can be included at a concentration of about 0.5 to about 10 μg/mL, about 1 to about 10 μg/mL, about 0.5 to about 5 μg/mL. or about 0.5 to about 2 μg/mL. In some embodiments, the culture medium comprises ascorbic acid at a concentration of about 0.5 μg/mL. In some embodiments, the culture medium comprises ascorbic acid at a concentration of about 1 μg/mL. In some embodiments, the culture medium comprises ascorbic acid at a concentration of about 5 μg/mL.

In some embodiments, the culture medium comprises heparin. Heparin can be included at a concentration of about 15 to about 30 μg/mL, about 20 to about 30 μg/mL, about 15 to about 25 g/mL. or about 20 to about 25 μg/mL. In some embodiments, the culture medium comprises ascorbic acid at a concentration of about 20 μg/mL. In some embodiments, the culture medium comprises ascorbic acid at a concentration of about 22.5 μg/mL. In some embodiments, the culture medium comprises ascorbic acid at a concentration of about 25 μg/mL.

In some embodiments, the culture medium comprises one antibiotics. Antibiotics may include, for example and without limitation, penicillin, streptomycin, gentamicin and amphotericin. In some embodiments, the culture medium comprises one or more of penicillin, streptomycin, gentamicin, amphotericin or any combination thereof.

In some embodiments, the culture medium comprises penicillin and streptomycin. In some embodiments, the culture medium comprises penicillin at an amount of about 1% by volume, about 2% by volume or about 5% by volume. In some embodiments, the culture medium comprises amphotericin at an amount of about 1% by volume, about 2% by volume or about 5% by volume. In some embodiments, the culture medium comprises penicillin and streptomycin at an amount of about 1% by volume.

In some embodiments, the culture medium comprises gentamicin and amphotericin. In some embodiments, the culture medium comprises gentamicin at an amount of about 25 μg/ml, 30 μg/ml, or about 35 μg/ml. In some embodiments, the culture medium comprises amphotericin at an amount of about 10 ng/ml, 15 ng/ml, or about 20 ng/ml. In some embodiments, the culture medium comprises gentamicin and amphotericin at a concentration of about 30 μg/ml gentamicin and about 15 ng/ml amphotericin.

In some embodiments, culture medium comprises one or more additional reagents selected from the group consisting of: serum, hydrocortisone, Epidermal Growth Factor (EGF), Insulin-like Growth Factor (IGF), an anticoagulant, and an antibiotic.

### PS-ECs and Cell Compositions

In another aspect, the present disclosure provides iPS-ECs cultured in accordance with methods described herein, including cell populations, cell lines thereof, and compositions or products comprising the same (“cell compositions”).

iPS-ECs cultured by use of compositions and methods described herein can form one or more of a monolayer, cell-cell contacts, and/or junctions (e.g., adherens junctions, tight junctions).

iPS-ECs cultured in accordance with methods of the present disclosure and cell compositions comprising the same can be or include an edited iPS-EC. Edited iPS-ECs have been modified to change at least one expression product of at least one gene at some point during the development and/or culturing of the cell. A modification can be introduced using methods known to a skilled artisan including, e.g., gene editing techniques such as CRISPR-Cas, Trancsription activator-like effector nucleases (TALENs), or Zinc Finger Nucleases (ZFNs). An iPS-EC cell can be edited (e.g., to produce an edited iPS-EC) at a time point before it has differentiated into an iPS-EC, e.g., at a precursor stage or stem cell stage and/or at a time point once the cell has been differentiated into an iPS-EC. An edited iPS-EC can be compared to a non-edited iPS-EC (an iPS-EC cell produced by differentiating a pluripotent stem cell in which the pluripotent stem cell and/or iPS-EC do not have modifications, e.g., genetic modifications).

In another aspect, the present disclosure provides iPS-ECs (e.g., cultured in accordance with methods described herein) and a culture medium comprising VEGF and bFGF. In the culture medium comprises about 30 to about 40 ng/mL VEGF. In some embodiments, the culture medium comprises about 20 to about 40 ng/mL bFGF.

In certain embodiments, the present disclosure comprises pharmaceutical compositions comprising iPS-ECs of the present disclosure or cell compositions described herein with a pharmaceutically acceptable carrier. Appropriate pharmaceutically acceptable carriers, include but are not limited to excipients and stabilizers, and are known in the art (see, e.g., Remington's Pharmaceutical Sciences (1990) Mack Publishing Co., Easton, PA). Pharmaceutically acceptable carriers may include, for example and without limitation, buffers, emulsifying agents, suspending agents, dispersing agents, isotonic agents, wetting agents, chelating agents, sequestering agents, pH buffer agents, antimicrobial agents, and/or antioxidants. Various excipients for formulating pharmaceutical compositions and techniques for preparing the composition are known in the art (see Remington: The Science and Practice of Pharmacy, 21st Edition, A. R. Gennaro, Lippincott, Williams & Wilkins, Baltimore, Md., 2006; incorporated herein by reference in its entirety). The use of a conventional excipient medium may be contemplated within the scope of the embodiments of the present disclosure, except insofar as any conventional excipient medium may be incompatible with a substance or its derivatives, such as by producing any undesirable biological effect or otherwise interacting in a deleterious manner with any other component(s) of the pharmaceutical composition.

In some embodiments, a pharmaceutical composition comprises a cell composition described herein comprising at least 50%, 60%, 70%, 80%, 90%, 95%, 98%, 99%, or 100% iPS-ECs (e.g., by weight of the total pharmaceutical composition). In some embodiments, a pharmaceutical composition comprises a cell composition described herein comprising about 95% to about 100% iPS-ECs (e.g., by weight of the total pharmaceutical composition).

In some embodiments, a pharmaceutical composition comprises iPS-ECs or a cell composition described herein that are allogeneic to a subject. In some embodiments, a pharmaceutical composition comprises iPS-ECs or a cell composition described herein that are autologous to a subject.

### Methods of Characterization

Methods of characterizing iPS-ECs cultured in accordance with technologies of the present disclosure can include characterization of one or more of (1) monolayer formation; (2) cell-cell contact formation; (3) junction formation (e.g., adherens junctions or tight junctions); and/or (4) functionality (e.g., cell growth/proliferation, metabolic consumption and/or production). Methods for characterizing monolayer formation, cell-cell contact formation, junction formation, and/or functionality are well known to those of skill in the art. Such methods include, but are not limited to microscopic methods (e.g., light microscopy, electron microscopy, fluorescent microscopy), immunocytochemistry and fluorescent microscopy, histological methods, electrophysiological recordings, impedance recording, flux assays, measurements of water permeability (Ussing chamber), transendothelial electrical resistance assays, proliferation assays, and/or metabolic profiling.

As will be understood by those of skill in the art, certain markers (e.g., for use with certain methods of characterization, such as immunocytochemistry and fluorescent microscopy) may be representative of cell-cell contact formation, junction formation, or cell growth/proliferation. For example, adherens junctions can be identified by, for example, the presence of cadherins and catenins, while tight junctions can be identified by, for example, the presence of occludin. Such markers are known to those of skill in the art.

### Uses

In one aspect, technologies of the present disclosure are useful in improved methods of culturing iPS-ECs. Such methods can provide a culture of iPS-ECs that form one or more of a monolayer, cell-cell contacts, and/or junctions (e.g., tight junctions, adherens junctions).

Methods of culturing iPS-ECs of the present disclosure and iPS-EC cells cultured in accordance with technologies of the present disclosure can be used in, for example, biomedical research. Such biomedical research can include, for example and without limitation, drug and/or compound therapeutic screening and/or testing, cytotoxicity screening, platelet or immune cell activation studies, and/or assessing vascular function (e.g., barrier function, thrombogenicity/non-thrombogenicity studies).

iPS-ECs cultured in accordance with technologies of the present disclosure can also be used in engineering vessels and/or organ grafts, including 3D printed grafts. iPS-ECs can be used to generate cellularized vasculature of grafts (e.g., 3D printed grafts) for both autologous and allogeneic applications. iPS-ECs can also function as a non-thromogenic barrier within, for example, arterial, venous, and capillaries of vasculature networks. It is understood monolayers of iPS-ECs can serve to, for example, prevent blood clot formation during blood flow and selectively enable or prohibit certain fluids, molecules, and/or proteins (e.g., cytokines) from moving outside the vasculature lumen

### Kits

In one aspect, the present disclosure provides kits comprising one or more containers comprising (i) a culture medium comprising one or more of factors selected from VEGF and bFGF; and (ii) instructions for use.

A container may include, for example and without limitation, a vial, well, test tube, flask, bottle, syringe, infusion bag, or other container means. Where an additional component (e.g., additional reagent, iPS-ECs) is included in the kit, the kit can contain additional containers into which this component may be placed. Containers and/or kits can comprise labeling with instructions for use and/or warnings.

In some embodiments, the culture medium comprises one or more additional reagents selected from the group consisting of: serum, hydrocortisone, Epidermal Growth Factor (EGF), Insulin-like growth factor (IGF), an anticoagulant, and an antibiotic. In some such embodiments, the or more additional reagents may be included in culture medium, or alternatively, may be in an additional container(s) comprising the one or more additional reagents to be added to the culture medium.

In some embodiments, the kit further comprises iPS-EC cells (e.g., for culturing in accordance with technologies described herein).

### EXAMPLES

### Example 1: Media Compatibility with iPS-ECs

The present example demonstrates use of culture medium comprising one or more growth factors selected from Vascular Endothelial Growth Factor (VEGF) and basic Fibroblast Growth Factor (bFGF) for culturing iPS-ECs.

Examples of media utilized in the present example is summarized in Table 1.1

TABLE 1.1
Examples of culture medium.

| Component | Media A | Media B | Media C |
|---|---|---|---|
| Media | Ncardia | Lonza | Lonza+ |
| Base media | HE-SFM (human | Basal Media | Basal Media |
| | endothelial serum | | |
| | free media) | | |
| Serum | Platelet-poor plasma | 2% FBS | 2% FBS |
| | derived serum | | |
| rhVEGF (ng/mL) | 30 | 0.5 | 30 |
| bFGF (ng/mL) | 20 | 10 | 20 |
| Hydrocortisone | N/A | 0.2 μg/mL | 0.2 μg/mL |
| rhEGF (ng/mL) | N/A | 5 | 5 |
| Long R3 IGF | N/A | 20 | 20 |
| (ng/mL) | | | |
| Ascorbic acid | N/A | 1 | 1 |
| (μg/mL) | | | |
| Heparin (μg/mL) | N/A | 22.5 | 22.5 |
| GA-1000 | 30 μg/mL | 30 μg/mL | 30 μg/mL |
| | Gentamicin | Gentamicin | Gentamicin |
| | 15 ng/mL | 15 ng/mL | 15 ng/mL |
| | Amphotericin | Amphotericin | Amphotericin |
| P/S | 0.5% | 1.0% | 1.0% |

iPS-ECs were plated on either discs or plastic, 400 μL of Media A, Media B, or Media C was added to each well, and the cells were incubated at 37° C. At 24 hours, 48 hours, 96 hours, and 168 hours metabolites were measured and the media was exchanged (old media aspirated and 500 μL of fresh media added). At 48 hours, 96 hours, and 168 hours, additional samples were fixed using 5% formalin for 10 minutes.

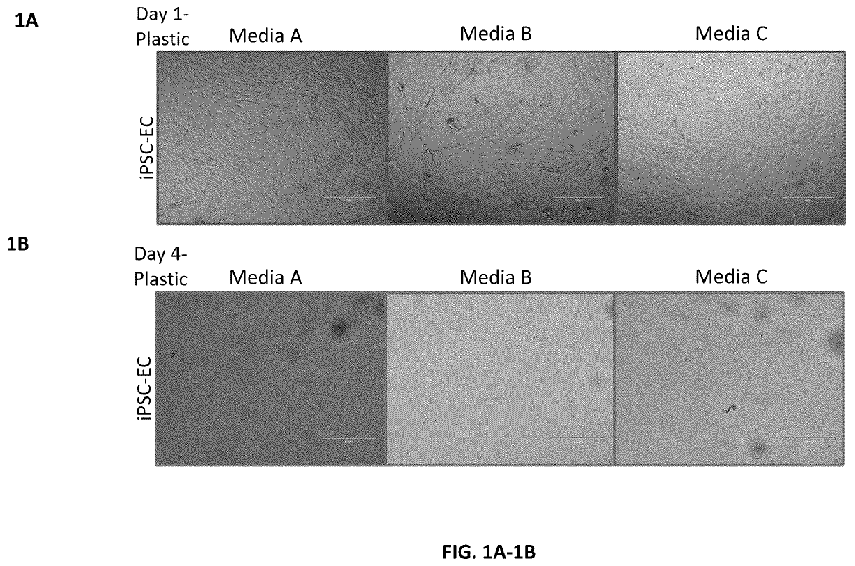

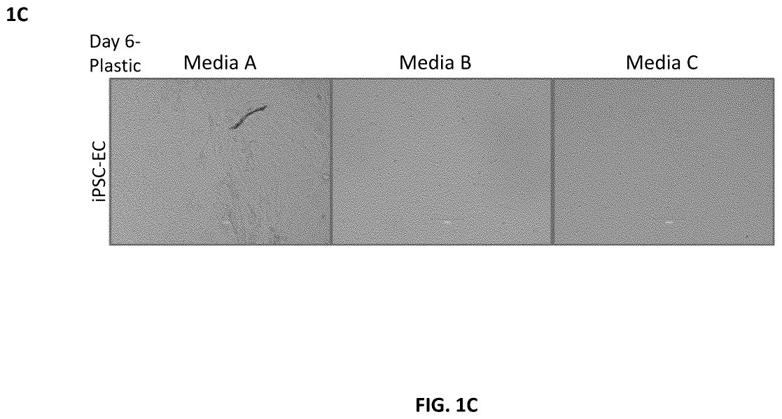

The low levels of VEGF and bFGF in Media B did not support attachment and morphology of the iPS-ECs. Growth of iPS-ECs in Media B led to cellular detachment at 24 hours post-plating (FIG. 1A), 96 hours post-plating (FIG. 1B), and 144 hours post-plating (FIG. 1C).

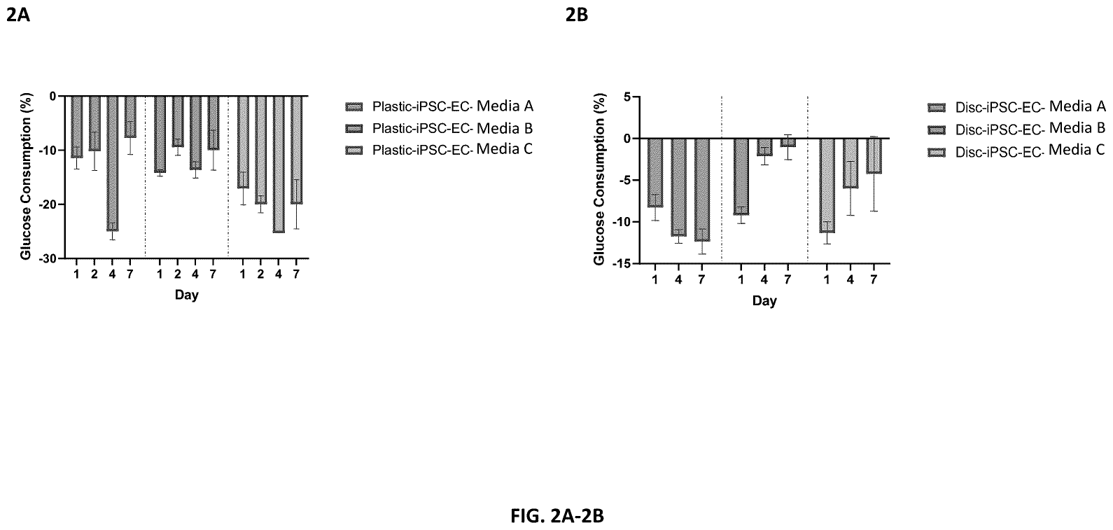

No significant difference for glucose consumption was observed between media groups with regards to iPS-ECs (plated on either plastic or discs). Significantly less glucose consumption was observed on the discs compared to the plastic for iPS-ECs (FIG. 2A-2B).

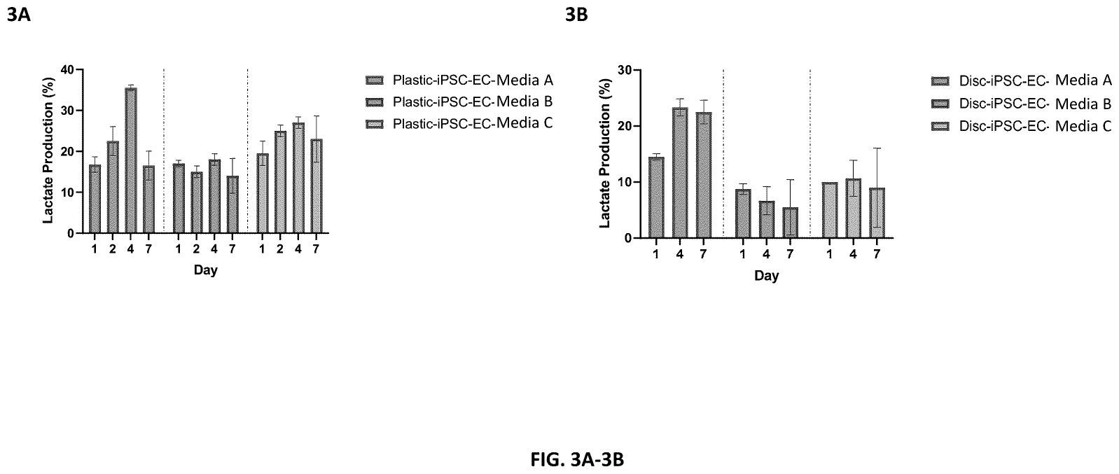

No significant difference was observed for lactate production between media groups with regards to the iPS-ECs. Significantly more lactate production was measured on the plastic compared to the discs for iPS-ECs (FIG. 3A-3B).

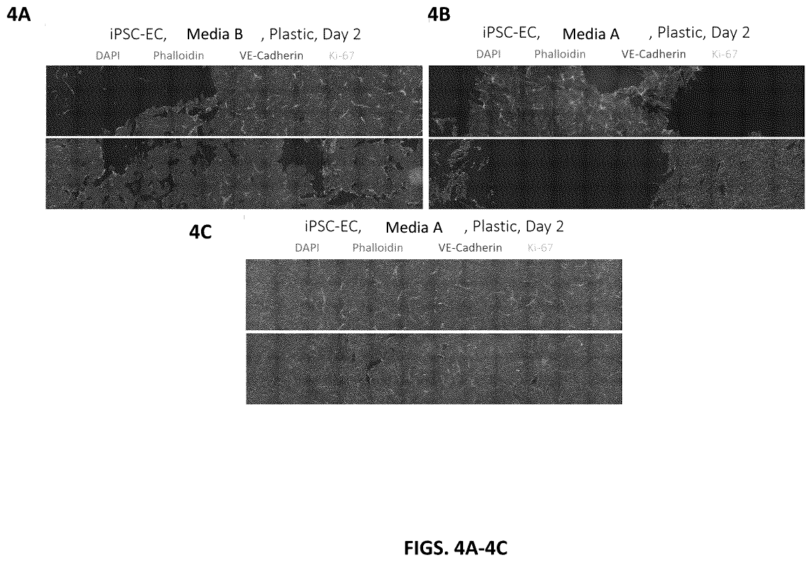

Fixed cells were stained with DAPI, Phalloidin, VE-Cadherin, and Ki-67. Results of iPS-EC from each media group plated on plastic are shown in FIGS. 4A-4C. FIG. 4C shows successful monolayer formation, while FIG. 4A-4B show a lack of successful monolayer formation on Day 2.

Results of the present example demonstrate iPS-ECs can grow in all of Medias A, B, and C. There was complete coverage for the iPSC-EC by Day 2 with Media C composition. The iPSC-ECs did not attach to the discs in any of the media conditions. iPSC-EC were dead or dying on the discs by day 4 on all conditions. The plastic demonstrates iPSC-ECs can grow in all 3 medias because they maintain the appropriate morphology, however time to form a monolayer and cell-cell contacts is shortest using Media C.

### Example 2: Comparison to Commercially-Available Media

The present Example demonstrates comparison of iPS-EC monolayer formation and cell-cell contact formation when cultured in Media C (as described in Example 1), Media D, or commercially available, Media E. Components of Media D and Media E are shown in Table 2.1.

TABLE 2.1
Examples of additional culture medias.

| | | Gibco Media 199 |
|---|---|---|
| Component | Media D | (Media E) |
| Media | Lonza++ | Gibco Media 199 |
| Base media | Basal Media | Gibco Media 199 |
| Serum | 10% FBS | 10% FBS |
| rhVEGF (ng/mL) | 40 | 40 |
| bFGF (ng/mL) | 40 | 40 |
| Hydrocortisone | 0.2 μg/mL | 0 |
| rhEGF (ng/mL) | 5 | 0 |
| Long R3 IGF (ng/mL) | 20 | 0 |
| Ascorbic acid (μg/mL) | 50 | 50 |
| Heparin (μg/mL) | 22.5 | 0 |
| GA-1000 | 500 | 0 |
| P/S | 1.0% | 1.0% |

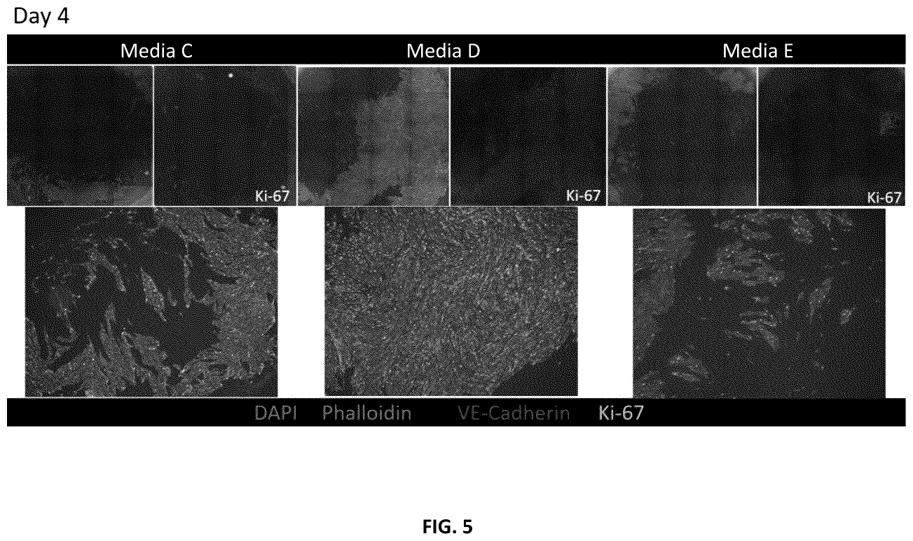

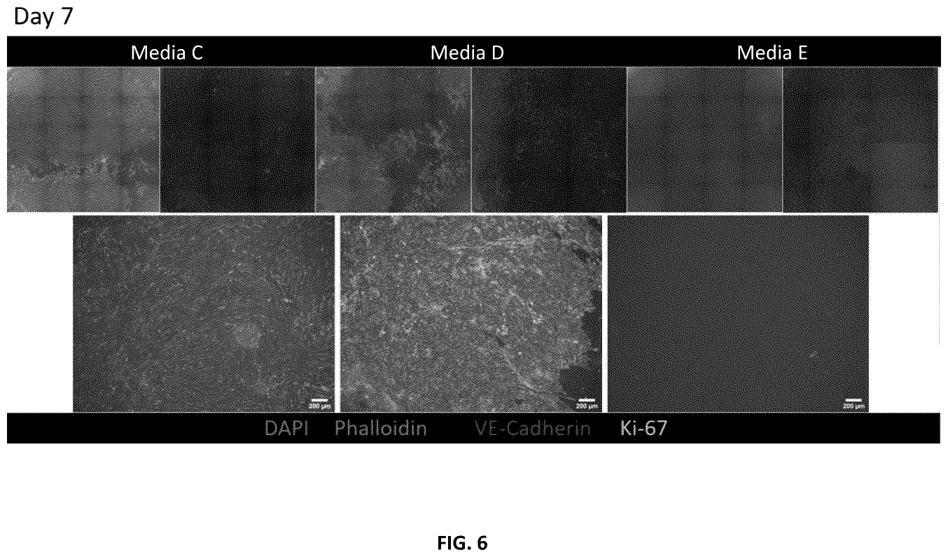

iPS-ECs were plated in either Media C, Media D, or Media E and cultured for up to 7 days. Cells were fixed after 4 and 7 days in culture and stained with DAPI (1:000 dilution), anti-Phalloidin antibody (1:400 dilution), anti-VE-Cadherein antibody (2 μg/mL), and anti-Ki-67 antibody (2 g/mL). Results demonstrated that the commercially available media, Media E, did not support the formation of iPS-EC monolayers and cell-cell contacts, while Media C and Media D supported both iPS-EC monolayer formation and cell-cell contact formation after 4 days (FIG. 5) and 7 days (FIG. 6). Without wishing to be bound by any one theory, it is hypothesized that Media E did not support the formation of iPS-EC monolayers and cell-cell contacts as Media E does not include hydrocortisone, rhEGF, long R3 IGF, or Heparin, which are understood to be required for iPS-EC cell growth and formation of monolayers and cell-cell contacts.

### Example 3: iPS-EC Cellularization of 3D Printed Rabbit Lobes

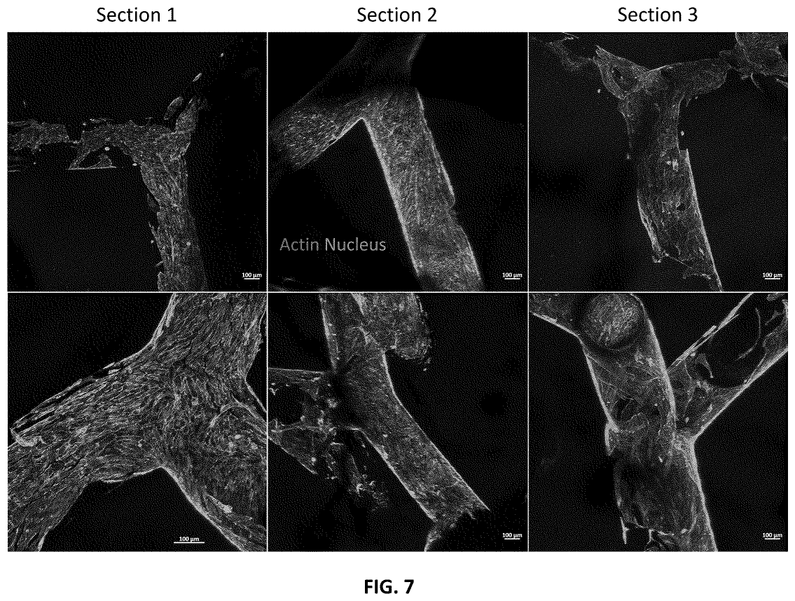

The present example demonstrates cellularization of 3D, bioink-printed rabbit lobes using iPS-ECs cultured utilizing Media C of the present disclosure. iPS-ECs were perfused into the rabbit lobes in order to cellularize the rabbit lobes. Cellularized rabbit lobes were then washed and perfused with 5% formalin for 10 minutes. Following formalin perfusion, the rabbit lobes were washed with PBS, permeabilized with 0.1% TritonX-100, and washed again. Rabbit lobes were then stained with DAPI (1:100) and phalloidin (1:40). Stained rabbit lobes were then cyrosectioned and imaged. FIG. 7 shows spreading and elongation of iPS-ECs seeded into rabbit lobes.

What is claimed is:

1. A method of culturing induced pluripotent stem cell derived endothelial cells (iPS-ECs) in vitro comprising culturing the iPS-ECs with a culture medium comprising one or more growth factors selected from Vascular Endothelial Growth Factor (VEGF) and basic Fibroblast Growth Factor (bFGF).

2. The method of claim 1, wherein the culture medium comprises about 30 to about 40 ng/mL VEGF.

3. The method of claim 1, wherein the culture medium comprises about 20 to about 40 ng/mL bFGF.

4. The method of claim 1, wherein the culture medium comprises one or more additional reagents selected from the group consisting of: serum, hydrocortisone, Epidermal Growth Factor (EGF), Insulin-like Growth Factor (IGF), an anticoagulant, and an antibiotic.

5. The method of claim 4, wherein the culture medium comprises serum at a concentration of about 2% by volume.

6. The method of claim 4, wherein the serum comprises fetal bovine serum.

7. The method of claim 4, wherein the culture medium comprises hydrocortisone at a concentration of about 0.2 μg/mL.

8. The method of claim 4, wherein the culture medium comprises EGF at a concentration of about 5 ng/mL.

9. The method of claim 4, wherein the culture medium comprises IGF at a concentration of about 20 ng/mL.

10. The method of claim 4, wherein the anticoagulant comprises heparin or ascorbic acid.

11. The method of claim 10, wherein the culture medium comprises ascorbic acid at a concentration of about 1 μg/mL.

12. The method of claim 10, wherein the culture medium comprises heparin at a concentration of about 22.5 μg/mL.

13. The method of claim 4, wherein the antibiotic comprises one or more of penicillin, streptomycin, gentamicin, amphotericin, or any combination thereof.

14. The method of claim 13, wherein the culture medium comprises penicillin and streptomycin at an amount of about 1% by volume.

15. The method of claim 13, wherein the culture medium comprises gentamicin and amphotericin at a concentration of about 30 g/ml gentamicin and about 15 ng/ml amphotericin.

16. The method of claim 1, wherein the culture medium comprises a base media for growing endothelial cells.

17. The method of claim 1, wherein the culture medium is exchanged with a frequency of about every day, every other day, every three days, every five days, or every week.

18. The method of claim 1, wherein the iPS-ECs are plated for culturing at a density of about 50,000 cells/cm², about 25,000 cells/cm², or about 12,500 cells/cm².

19. The method of claim 1, wherein the iPS-ECs form one or more of a monolayer, cell-cell contacts, and tight junctions.

20. An iPS-EC culture comprising iPS-ECs and a culture medium comprising VEGF and bFGF.

21. The composition of claim 20, wherein the culture medium comprises about 30 to about 40 ng/mL VEGF.

22. The composition of claim 20, wherein the culture medium comprises about 20 to about 40 ng/mL bFGF.
